SEQUENCE LISTING (26 sequences)

SEQ ID NO: 1 - DNA, 23 bp, synthetic construct
  misc_feature 1..23: note = Synthetic Sequence
  source 1..23: mol_type = other DNA
gaggattttgctaacctgacacc

SEQ ID NO: 2 - DNA, 21 bp, synthetic construct
  misc_feature 1..21: note = Synthetic Sequence
  source 1..21: mol_type = other DNA
ttgacggtaactgactccagc

SEQ ID NO: 3 - DNA, 21 bp, synthetic construct
  misc_feature 1..21: note = Synthetic Sequence
  source 1..21: mol_type = other DNA
cccaaagctaaccgggagaag

SEQ ID NO: 4 - DNA, 20 bp, synthetic construct
  misc_feature 1..20: note = Synthetic Sequence
  source 1..20: mol_type = other DNA
ccagaatccaacacgatgcc

SEQ ID NO: 5 - DNA, 21 bp, synthetic construct
  misc_feature 1..21: note = SYnthetic Sequence
  source 1..21: mol_type = other DNA
gtcccagacatcagggagtaa

SEQ ID NO: 6 - DNA, 21 bp, synthetic construct
  misc_feature 1..21: note = Synthetic Sequence
  source 1..21: mol_type = other DNA
tcggatacttcagcgtcagga

SEQ ID NO: 7 - DNA, 19 bp, synthetic construct
  misc_feature 1..19: note = Synthetic Sequence
  source 1..19: mol_type = other DNA
gtgccaggatgtgtgacga

SEQ ID NO: 8 - DNA, 21 bp, synthetic construct
  misc_feature 1..21: note = Synthetic Sequence
  source 1..21: mol_type = other DNA
ctgtcccatacccaccatgac

SEQ ID NO: 9 - DNA, 21 bp, synthetic construct
  misc_feature 1..21: note = Synthetic Sequence
  source 1..21: mol_type = other DNA
gaggtcactcctatcctctgg

SEQ ID NO: 10 - DNA, 22 bp, synthetic construct
  misc_feature 1..22: note = Synthetic Sequence
  source 1..22: mol_type = other DNA
gccatttcctccgacttttctc

SEQ ID NO: 11 - DNA, 20 bp, synthetic construct
  misc_feature 1..20: note = Synthetic Sequence
  source 1..20: mol_type = other DNA
tgcaaaggctccaggtctga

SEQ ID NO: 12 - DNA, 21 bp, synthetic construct
  misc_feature 1..21: note = Synthetic Sequence
  source 1..21: mol_type = other DNA
cttgaacctgtccaaccacaa

SEQ ID NO: 13 - DNA, 19 bp, synthetic construct
  misc_feature 1..19: note = Synthetic Sequence
  source 1..19: mol_type = other DNA
ctggcggttcaggtccaat

SEQ ID NO: 14 - DNA, 19 bp, synthetic construct
  misc_feature 1..19: note = Synthetic Sequence
  source 1..19: mol_type = other DNA
ttccaggcaatccacgagc

SEQ ID NO: 15 - DNA, 21 bp, synthetic construct
  misc_feature 1..21: note = Synthetic Sequence
  source 1..21: mol_type = other DNA
ccccagtgtccttacagagtg

SEQ ID NO: 16 - DNA, 19 bp, synthetic construct
  misc_feature 1..19: note = Synthetic Sequence
  source 1..19: mol_type = other DNA
gtgcccagagtggatgtct

SEQ ID NO: 17 - DNA, 22 bp, synthetic construct
  misc_feature 1..22: note = Synthetic Sequence
  source 1..22: mol_type = other DNA
gcaactgttcctgaactcaact

SEQ ID NO: 18 - DNA, 21 bp, synthetic construct
  misc_feature 1..21: note = Synthetic Sequence
  source 1..21: mol_type = other DNA
atcttttggggtccgtcaact

SEQ ID NO: 19 - DNA, 23 bp, synthetic construct
  misc_feature 1..23: note = Synthetic Sequence
  source 1..23: mol_type = other DNA
tagtccttcctaccccaatttcc

SEQ ID NO: 20 - DNA, 21 bp, synthetic construct
  misc_feature 1..21: note = Synthetic Sequence
  source 1..21: mol_type = other DNA
ttggtccttagccactccttc

SEQ ID NO: 21 - DNA, 22 bp, synthetic construct
  misc_feature 1..22: note = SYnthetic Sequence
  source 1..22: mol_type = other DNA
gaagaagagcgaaggaaaaggc

SEQ ID NO: 22 - DNA, 21 bp, synthetic construct
  misc_feature 1..21: note = Synthetic Sequence
  source 1..21: mol_type = other DNA
gcatcaggataagctgttgcc

SEQ ID NO: 23 - DNA, 19 bp, synthetic construct
  misc_feature 1..19: note = Synthetic Sequence
  source 1..19: mol_type = other DNA
cctggccctgctgaacttg

SEQ ID NO: 24 - DNA, 19 bp, synthetic construct
  misc_feature 1..19: note = Synthetic Sequence
  source 1..19: mol_type = other DNA
gacgtgggtcatcaccgat

SEQ ID NO: 25 - DNA, 22 bp, synthetic construct
  misc_feature 1..22: note = Synthetic Sequence
  source 1..22: mol_type = other DNA
acaaggaattgaatgacctggc

SEQ ID NO: 26 - DNA, 19 bp, synthetic construct
  misc_feature 1..19: note = Synthetic Sequence
  source 1..19: mol_type = other DNA
caccctgataggggctgtc

(19) United States
(12) Patent Application Publication
(10) Pub. No.: US 2023/0141929 A1
(43) Pub. Date: May 11, 2023

(54) METHOD FOR TREATING CARDIOVASCULAR DISEASE

(71) Applicant: TECHNION RESEARCH & DEVELOPMENT FOUNDATION LIMITED, Haifa (IL)

(72) Inventors: Ami ARONHEIM, Binyamina (IL); Roy KALFON, Haifa (IL); Lilach KOREN, Haifa (IL)

(21) Appl. No.: 18/080,049

(22) Filed: Dec. 13, 2022

Related U.S. Application Data
(63) Continuation of application No. 17/053,896, filed on Nov. 9, 2020, now abandoned.
(60) Provisional application No. 62/674,089, filed on May 21, 2018.

(51) Int. Cl. C12N 15/113; A61P 9/04

(52) U.S. Cl. CPC C12N 15/1138; A61P 9/04; C12N 2310/11; C12N 2310/12; C12N 2310/14; C12N 2310/141; C12N 2310/15

(57) ABSTRACT

The invention relates to a method of treating a cardiovascular disease, such as heart failure, in a subject in need comprising the step of administering an inhibitor of bZIP repressor or an activator of p38 or a combination thereof to a subject in need thereby treating the cardiovascular disease. The inhibitor to bZIP repressor is: an inhibitor of ATF3; an inhibitor of JDP2; a co-inhibitor to both ATF3 and JDP2; or a combination of an inhibitor of ATF3 and an inhibitor of JDP2.

### CROSS REFERENCE TO RELATED APPLICATIONS

This application is a Continuation of U.S. patent application Ser. No. 17/053,896, filed on Nov. 9, 2020, which is a National Phase of PCT Patent Application No. PCT/IL2019/050566 having International filing date of May 19, 2019, which claims the benefit of priority of U.S. Provisional Application No. 62/674,089 filed on May 21, 2018. The contents of the above applications are all incorporated by reference as if fully set forth herein in their entirety.

### REFERENCE TO AN ELECTRONIC SEQUENCE LISTING

The contents of the electronic sequence listing (TECH_010-US1.xml; Size: 33,906 bytes; and Date of Creation: Jan. 17, 2023) is herein incorporated by reference in its entirety.

### BACKGROUND OF THE INVENTION

Heart failure (HF), also known as chronic heart failure (CHF), is when the heart is unable to pump sufficiently to maintain blood flow to meet the body's needs. Signs and symptoms of heart failure commonly include shortness of breath, excessive tiredness, and leg swelling. The shortness of breath is usually worse with exercise, while lying down, and may wake the person at night. A limited ability to exercise is also a common feature. Chest pain, including angina, does not typically occur due to heart failure

The severity of disease is graded by the severity of symptoms with exercise. Heart failure is not the same as myocardial infarction (in which part of the heart muscle dies) or cardiac arrest (in which blood flow stops altogether).

Treatment depends on the severity and cause of the disease. In people with chronic stable mild heart failure, treatment commonly consists of lifestyle modifications such as stopping smoking, physical exercise] and dietary changes, as well as medications.

ACE inhibitors lower blood pressure and reduce strain on the heart. They also may reduce the risk of a future heart attack. Aldosterone antagonists trigger the body to remove excess sodium through urine. This lowers the volume of blood that the heart must pump. Angiotensin receptor blockers relax the blood vessels and lower blood pressure to decrease the heart's workload. Beta blockers slow the heart rate and lower the blood pressure to decrease the heart's workload. Digoxin makes the heart beat stronger and pump more blood. Diuretics (fluid pills) help reduce fluid buildup in the lungs and swelling in the feet and ankles.

Isosorbide dinitrate/hydralazine hydrochloride helps relax the blood vessels so the heart doesn't work as hard to pump blood. Studies have shown that this medicine can reduce the risk of death in blacks. More studies are needed to find out whether this medicine will benefit other racial groups.

Myocardial infarction (MI) is a life-threatening event and may cause cardiac sudden death or heart failure. Despite considerable advances in the diagnosis and treatment of heart disease, cardiac dysfunction after MI is still the major cardiovascular disorder that is increasing in incidence, prevalence, and overall mortality). After acute myocardial infarction, the damaged cardiomyocytes are gradually replaced by fibroid nonfunctional tissue. Ventricular remodeling results in wall thinning and loss of regional contractile function. The ventricular dysfunction is primarily due to a massive loss of cardiomyocytes. It is widely accepted that adult cardiomyocytes have little regenerative capability.

Therefore, the loss of cardiac myocytes after MI is irreversible. Each year more than half million Americans die of heart failure. The relative shortage of donor hearts forces researchers and clinicians to establish new approaches for treatment of cardiac dysfunction in MI and heart failure patients.

All currently available drugs to both MI and heart failure aim to reduce blood pressure or to reduced fluid load. There is a need to target the cardiomyocytes in order to obtain better contractile function and suppress remodeling processes due to pressure overload and heart failure.

### SUMMARY OF THE INVENTION

In some embodiments of the invention, there is provided a method of treating a cardiovascular disease in a subject in need comprising the step of administering an inhibitor of bZIP repressor or an activator of p38 or a combination thereof to a subject in need thereby treating the cardiovascular disease.

The inhibitor to bZIP repressor is in some embodiments of the invention:
an inhibitor of ATF3;
an inhibitor of JDP2;
a co-inhibitor to both ATF3 and JDP2; or
a combination of an inhibitor of ATF3 and an inhibitor of JDP2.

In some embodiments of the invention, the cardiovascular disease is heart failure.

In some embodiments of the invention, the cardiovascular disease is accompanied by maladaptive cardiac remodeling process.

In some embodiments of the invention, the cardiovascular disease is accompanied by reduced contractile function.

In some embodiments of the invention, the treating is effected by improvement of the contraction of the cardiomyocyte.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1 A, B and C demonstrate that dKO male mice display attenuated cardiac hypertrophy following TAC. Male mice were treated with TAC for 8 weeks and their hearts were analyzed. FIG. 1A demonstrates representative pictures of control and TAC-operated mice hearts of each genotype. The percentage increase in ventricles weight (VW) to mouse body weight (BW) ratio (mg/gr) by TAC is shown at the bottom. FIG. 1B shows the ratio of VW/BW (n=7-15/group). FIG. 1C show expression levels of mRNA that are presented as relative values (compared to wild type control mice, defined as 1, n=6-8/group). mRNA was extracted from ventricles and the expression level of cardiac remodeling, hypertrophic and inflammatory markers were measured by qRT-PCR. All results represent the mean SE ***P≤0.05, control vs. TAC; ^{†}P≤0.05, difference between genotypes.

FIGS. 2 A, B, C and D demonstrate that dKO male mice display attenuated cardiac fibrosis following TAC. Male mice were treated with TAC for eight weeks and their hearts were analyzed. FIG. 2A demonstrate photographs of ventricles sections that were stained with FITC-labeled wheat germ agglutinin and cell size was analyzed. Scale bar=100 μm. FIG. 2B shows the quantification of cell size from D represented as cross sectional area in m². FIG. 2C is a photograph of representative paraffin-embedded heart sections stained with Masson's trichrome to visualize fibrosis. FIG. 2D shows the quantification of the level of fibrosis (%) stained by Masson's trichrome (n=6-8/group). All results represent the mean SE ***P 50.05, control vs. TAC; ^{†}P≤0.05, difference between genotypes.

FIGS. 3 A, B and C show that dKO male mice display increased p38 activity. Cardiac hypertrophy was induced by TAC in male mice. Eight weeks following TAC, mice were sacrificed and hearts were excised FIG. 3A Western blot analysis of heart lysate derived from the indicated genotypes with the indicated antibodies. FIG. 3B and FIG. 3C show the densitometric analysis of Western blot shown in Figure A presented as mean ratio of the corresponding phospho-protein to total protein f SE (compared to wild type control, defined as 1, n=5-6/group). B pp38/p38. C pERK/ERK. ***P≤0.05, control vs. TAC; ^{†}P≤0.05 difference between genotypes.

FIGS. 4 A, B and C show that dKO male mice preserve contractile function following TAC. Cardiac hypertrophy was induced by TAC in male mice. Eight weeks following TAC, left ventricular cardiac volumes, mass and function were examined by a cardiac MRI. FIG. 4A is a table demonstrating the following parameters that were measured: left ventricular (LV) mass, left ventricular end-diastolic (LVEDV) and left ventricular end-systolic volume (LVESV), and ejection fraction (EF) was calculated. The results represent the mean±SE of the indicated number (n) of animals per group. ***P≤0.05, control vs. TAC; ^{†}P≤0.05, difference between genotypes. FIG. 4B is representative images of mid-ventricular short-axis slice at peak diastole and systole. FIG. 4C is a table showing age-related decline in cardiac function as was assessed at 50- and 80-weeks-old mice. Results were compared with control mice (20 weeks old). Left ventricular cardiac volumes, mass and function were examined by a cardiac MRI as described in FIG. 4A. The results represent the mean±SE of the indicated number (n) of animals per group. ***P≤0.05, control vs. TAC; ^{†}P≤0.05, difference between genotypes. ***P≤0.05, different from 20- and 50-weeks-old mice; ^{†}P≤0.05, difference between genotypes.

FIG. 5 is a schematic diagram showing the dual loss of ATF3 and JDP2 model in cardiac remodeling. The interplay between JDP2 and ATF3 is shown in various mouse strains used in this and previous manuscript and the cardiac consequences in maintaining heart function in health (left panels) and following TAC (right panels). JDP2 and ATF3 protein expression levels are represented by black and light-blue circles, respectively. Other stress induced proteins are shown in red ovals. The panels represent the following mice strains: WT, ATF3 KO, JDP2 KO and dKO. Color code scale bar representing cardiac remodeling from adaptive to maladaptive is shown at the bottom (white to dark-grey respectively).

FIGS. 6 A and B are graphs showing that dKO male mice display attenuated cardiac hypertrophy following TAC. Male mice were treated with TAC for 8 weeks and their hearts were analyzed. FIG. 6A presents the Mice body weight (BW). FIG. 6B presents mice ventricles weight (VW). All results represent the mean SE. ***P≤0.05, control vs. TAC; ^{†}P≤0.05, difference between genotypes.

FIGS. 7 A, B, C, D, E and F are graphs showing that dKO female mice display reduced cardiac hypertrophy and fibrosis following TAC. Cardiac hypertrophy was induced by TAC in female mice. Eight weeks following TAC, mice were sacrificed and hearts were excised. FIG. 7A shows that the ratio (mg/gr) of ventricles weigh (VW) to mouse body weight (BW) VW/BW (mg/gr) is shown. FIG. 7B shows mice BW. FIG. 7C shows mice VW. FIG. 7D shows the expression level of mRNA that was extracted from ventricles and the expression level of cardiac remodeling and hypertrophic, fibrosis and inflammatory markers that were measured by qRT-PCR. Expression levels are presented as relative values (compared to wild type control mice, defined as 1, n=6-8/group). FIG. 7E shows the quantification of cross-sectional area in μm² of ventricles sections that were stained with FITC-labeled wheat germ agglutinin. FIG. 7F shows quantification of paraffin-embedded heart sections that were stained with Masson's trichrome to visualize fibrosis and the level of fibrosis (%) as quantified (n=6-8/group). All results represent the mean±SE ***P≤0.05, control vs. TAC; ^{†}P≤0.05, difference between genotypes.

FIG. 8 is a table showing that dKO female mice preserve contractile function following TAC. Cardiac hypertrophy was induced by TAC in female mice. Eight weeks following TAC, mice hearts were examined by micro ultrasound. The following parameters were measured: interventricular septal end diastole (IVSd); left ventricular posterior wall end diastole (LVPWd); maximal left ventricular internal end-diastole (LVIDd); end-systole (LVIDs); and fractional shortening (FS). FS was assessed according to: FS (%)=[(LVDd−LVDs)/LVDd]*100. All results represent the means±SE of the indicated number (n) of animals per group. ***P≤0.05, control vs. TAC; ^{†}P≤0.05, difference between genotypes.

### DETAILED EMBODIMENTS OF THE INVENTION

c-Jun dimerization protein (JDP2) and Activating Transcription Factor 3 (ATF3) are closely related basic leucine zipper proteins. Transgenic mice with cardiac expression of either JDP2 or ATF3 showed maladaptive remodeling and cardiac dysfunction. Surprisingly, JDP2 knockout (KO) did not protect the heart following transverse aortic constriction (TAC). Instead, the JDP2 KO mice performed worse than their wild type (WT) counterparts. To test whether the maladaptive cardiac remodeling observed in the JDP2 KO mice is due to ATF3, ATF3 was removed in the context of JDP2 deficiency, referred as double KO mice (dKO). Mice were challenged by TAC, and followed by detailed physiological, pathological and molecular analyses. dKO mice displayed no apparent differences from WT mice under unstressed condition, except a moderate better performance in dKO male mice. Importantly, following TAC the dKO hearts showed low fibrosis levels, reduced inflammatory and hypertrophic gene expression and a significantly preserved cardiac function as compared with their WT counterparts in both genders. Consistent with these data, removing ATF3 resumed p38 activation in the JDP2 KO mice which correlates with the beneficial cardiac function. Collectively, mice with JDP2 and ATF3 double deficiency had reduced maladaptive cardiac remodeling and lower hypertrophy following TAC. As such, the worsening of the cardiac outcome found in the JDP2 KO mice is due to the elevated ATF3 expression. Simultaneous suppression of both ATF3 and JDP2 activity is highly beneficial for cardiac function in health and disease.

JDP2 and ATF3 are bZIP transcription factors that share 90% homology in their bZIP region. Both proteins can form heterodimers with other bZIP family members and can either suppress or activate transcription as homodimers or heterodimers in a context-dependent manner. A key difference between them is their bioavailability and mode of regulation. Whereas JDP2 is ubiquitously expressed, ATF3 is an immediate-early gene that is normally expressed at a low or undetectable level, but is highly induced by numerous stress signals. Interestingly, these proteins regulate the expression of each other. Therefore, deficiency in either one of them results in an elevated expression of the other gene. Thus far, each gene has been shown to play a role in a variety of pathophysiological contexts using various mouse disease models such as cancer, neurodegeneration, diabetes, atherosclerosis, and heart failure. Among these, cardiac disease is a model that has been used to investigate JDP2 and ATF3. Using a gain-of-function approach, it was shown that transgenic mice ectopically expressing either JDP2 or ATF3 displayed maladaptive cardiac remodeling and hypertrophy. The effects were independent of developmental events, since hypertrophic cardiac growth was observed following expression in adult mice using an inducible tet-off system. Further their roles in the heart using a loss-of-function approach was investigated.

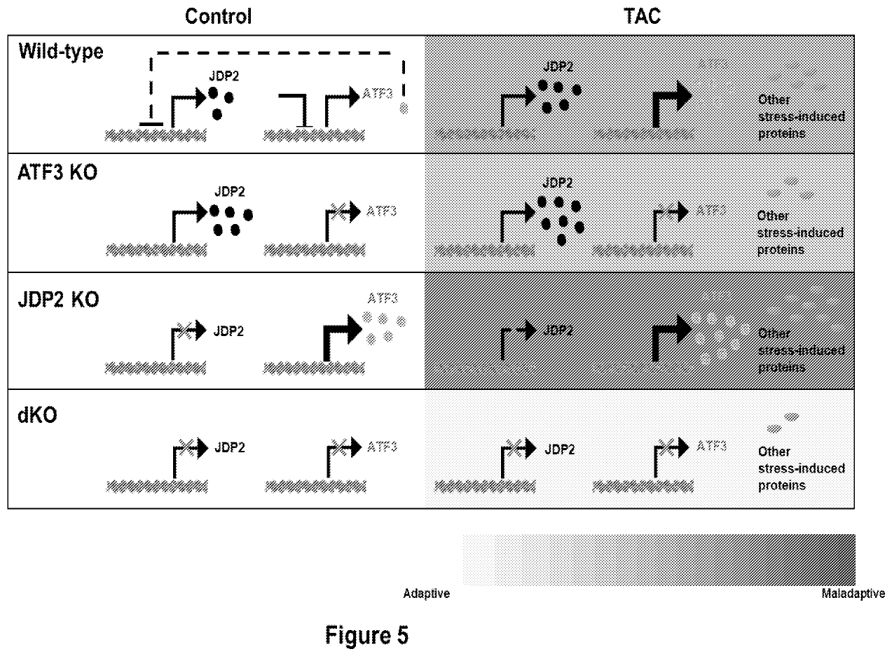

Consistent with the detrimental role of ATF3, its deletion afforded partial cardiac protection in the ATF3 KO mice in phenylephrine infusion model, while in the TAC model, ATF3 had a very mild beneficial outcome compared with WT mice. In contrast, JDP2 deletion resulted in deterioration of cardiac function following TAC. A possible explanation for this discrepancy is that JDP2 overexpression mimics ATF3 function due to their high sequence homology. On the other hand, JDP2 deficiency results in elevated expression of ATF3, which was previously shown to promote cardiac maladaptive remodeling as well. Therefore, both JDP2 overexpression and deficiency results in a net elevation of bZIP repressor activity. This may alter the delicate equilibrium between numerous bZIP family members resulting in a deteriorated outcome. Indeed, in the study it was demonstrated that JDP2 KO mice lacking ATF3 display improved cardiac outcome with preserved contractile function, supporting the above hypothesis. These results were observed in both male and female dKO mice and were significantly different than the expected additive mixed single KO genotypes. The interplay between JDP2 and ATF3 single KOs and dKO and their role in cardiac adaptation or maladaptation under stress is summarized (FIG. 5). dKO mice display a better outcome in all molecular and physiological parameters used to assess cardiac remodeling and hypertrophy. This include hypertrophic markers, fibrosis, immune response, and cardiac function. Importantly, when the calculated EF for all four genotypes namely; ATF3 KO and JDP2 KO mice from a previous article (Kalfon et al. Int J Cardiol. 2017; 249:357-363) were compared to WT and dKO mice following TAC, the EF of dKO mice is significantly better than the EF of the single KOs of both ATF3 KO and JDP2 KO mice and is similar to the EF representing un-operated WT mice.

Since the dKO mice are deficient of JDP2 and ATF3 upon fertilization, one caveat is that the improved cardiac performance is due to some yet unidentified developmental beneficial effects, rather than better adaptation to the TAC stress. To address this issue, the mice were analyzed under un-stressed condition. In dKO male mice displayed higher VW/BW ratio than the WT mice. The higher VW/BW ratio in males is due to lower BW and is not observed in female mice. Functionally, dKO mice showed improved cardiomyocyte contractile function when compared with WT mice in both gender. This improvement was sustained in older mice at 50 and 80 weeks of age as well. In contrast, in the females VW/BW ratio, cardiac function and sarcomeric actin levels were indistinguishable between the genotypes; yet, following TAC, the dKO females displayed a cardiac protective phenotype. Thus, the beneficial phenotype that was observed following TAC in the dKO mice is independent of their basal cardiac function, making it unlikely to exhibit cardiac protection due to some unspecified developmental benefits.

It is noted that, in an apparent contradiction, two studies showed that ATF3 deficiency resulted in a deteriorated phenotype under TAC. The mice were examined at 8 weeks post TAC, while the others at 4 weeks. It is well known that cardiac stress initially induces an adaptive response aiming to preserve cardiac function; however, when stress becomes chronic, the adaptive process turns into a maladaptive one. This fits well with the current understanding of the ATF3 biology. ATF3 is a stress gene induced by a long list of signals that disturb cellular homeostasis. On the one hand, its induction under acute conditions appears to be beneficial, facilitating the cells to adapt. On the other hand, its expression under chronic conditions almost invariably leads to pathological consequences. As an example, acute induction of ATF3 in the pancreatic beta cells upon exposure to glucose increases their ability to up-regulate insulin gene expression and subsequent secretion. However, chronic induction of ATF3 leads to beta cell apoptosis. Thus, the potential dichotomous role of ATF3 under acute versus chronic stress may be an explanation for the apparent discrepancy in the literature (above).

Both JDP2 and ATF3 are transcription factors. Clearly, an important mechanistic question is “what are the functionally relevant downstream targets for ATF3 and JDP2 in the context of cardiac stress?” It appears that the activity of the p38 signaling pathway plays a significant role and positively correlates with the cardiac function. Previously, it was shown that the p38 pathway was completely abrogated in JDP2 deficient mice following TAC (See Kalfon et al. Int J Cardiol. 2017; 249:357-363). However, the present study showed a resumption of the p38 activation in the dKO mice. In addition, the level of p38 activation in the dKO mice was higher than that in the WT mice with or without TAC, and is correlated with the beneficial cardiac outcomes.

Although much advance is made through the use of genetically modified mice, compensatory mechanisms can obscure interpretation and may not truly represent the functional role of the targeted molecule. The identification of such compensatory mechanisms in the future is crucial for better understanding the complex interplay between key regulatory molecules.

In summary, it is suggested that JDP2 and ATF3 double deficiency correlates positively with p38 activation and afforded a beneficial cardiac effect in both genders in response to pressure overload. Current treatments for heart failure are very limited. The inhibition of both JDP2 and ATF3, or the activation of p38 in the heart may serve as promising means to reduce maladaptive cardiac remodeling and improve cardiac function.

In an embodiment of the invention, there is provided a method of treating a cardiovascular disease in a subject in need comprising the step of administering an inhibitor of bZIP repressor or an activator of p38 or a combination thereof to a subject in need thereby treating the cardiovascular disease.

In some embodiments of the invention, the inhibitor to bZIP repressor is:
an inhibitor of ATF3;
an inhibitor of JDP2;
a co-inhibitor to both ATF3 and JDP2; or
a combination of an inhibitor of ATF3 and an inhibitor of JDP2.

In some embodiments of the invention, the inhibitor to ATF3 and the inhibitor to JDP2 are administered simultaneously or sequentially.

In some embodiments of the invention, the inhibitor is a protein, a peptide, a small molecule or an agent, which prevents or reduces the expression of the bZIP repressor.

In some embodiments of the invention, the activator of p38 is a protein, a peptide, a small molecule or an agent, which increases the expression of the p38.

In some embodiments of the invention, the agent which decreases the expression of the bZIP repressor is an inhibitor of the mRNA encoding the bZIP repressor.

In some embodiments of the invention, the inhibitor of the mRNA encoding the bZIP repressor is an antisense RNA, triple helix molecule, ribozyme, microRNA, or siRNA that recognizes the bZIP repressor mRNA.

In some embodiments of the invention, the agent which increases the expression of the p38 is an mRNA encoding the p38 or an activator thereof.

In some embodiments of the invention, the activator of the mRNA encoding the p38 or the activator thereof is an antisense RNA, triple helix molecule, ribozyme, microRNA, or siRNA that recognizes the bZIP repressor mRNA.

In some embodiments of the invention, wherein the cardiovascular disease is heart failure.

In some embodiments of the invention, the heart failure is a chronic heart failure (CHF).

In some embodiments of the invention, the cardiovascular disease is accompanied by maladaptive cardiac remodeling process.

In some embodiments of the invention, the cardiovascular disease is accompanied by reduced contractile function.

In some embodiments of the invention, the cardiovascular disease is accompanied by maladaptive cardiac remodeling process.

In some embodiments of the invention, the cardiovascular disease is accompanied by reduced contractile function.

In some embodiments of the invention, the treating is effected by improvement of the contraction of the cardiomyocyte.

As used herein, the term “cardiomyocyte” refers to any cell in the cardiac myocyte lineage that shows at least one phenotypic characteristic of a cardiac muscle cell. Such phenotypic characteristics can include expression of cardiac proteins, such as cardiac sarcomeric or myofibrillar proteins or atrial natriuretic factor, or electrophysiological characteristics. As used herein, the term “cardiomyocyte” and “myocyte” are interchangeable.

As used herein, the term “heart failure” refers to the loss of cardiomyocytes such that progressive cardiomyocyte loss over time leads to the development of a pathophysiological state whereby the heart is unable to pump blood at a rate commensurate with the requirements of the metabolizing tissues or can do so only from an elevated filling pressure. The cardiomyocyte loss leading to heart failure may be caused by apoptotic mechanisms.

In some embodiments of the invention the subject in need thereof has a damaged myocardium.

In some embodiments of the invention the subject in need thereof is diagnosed with or suffering from heart failure.

In some embodiments of the invention the subject in need thereof is diagnosed with or suffering from an age-related cardiomyopathy.

In some circumstances, one or more symptoms associated with cardiovascular diseases, e.g., heart failure, myocardial infarction, an age-related cardiomyopathy or a damaged myocardium, can be reduced or alleviated following administration of the inhibitors to bZIP repressor and in particular from a combined treatment with an inhibitor of ATF3 and an inhibitor of JDP2. Symptoms of heart failure include, but are not limited to, fatigue, weakness, rapid or irregular heartbeat, dyspnea, persistent cough or wheezing, edema in the legs and feet, and swelling of the abdomen. Symptoms for myocardial infarction include, but are not limited to, prolonged chest pain, heart palpitations (i.e. abnormality of heartbeat), shortness of breath, and extreme sweating. Non-limiting symptoms of an age-related cardiomyopathy, e.g., restrictive cardiomyopathy, include coughing, difficulty breathing during normal activities or exercise, extreme fatigue, and swelling in the abdomen as well as the feet and ankles.

In some embodiments of the invention, the treatment of the invention is considered to be pharmaceutically effective if the dosage alleviates at least one symptom of cardiovascular disease described above by at least about 10%, at least about 15%, at least about 20%, at least about 30%, at least about 40%, or at least about 50%. In one embodiment, at least one symptom is alleviated by more than 50%, e.g., at least about 60%, or at least about 70%. In another embodiment, at least one symptom is alleviated by at least about 80%, at least about 90% or greater, as compared to a subject having the same disease that was not treated by an inhibitor of bZIP repressor and in particular was not treated by a combination of an inhibitor to ATF3 and an inhibitor to JDP2.

In some embodiments of the invention, the treatment of the invention is considered to be pharmaceutically effective if the dosage alleviates the cardiomyocytes contractile function in at least about 10%, at least about 15%, at least about 20%, at least about 30%, at least about 40%, or at least about 50%. In one embodiment, the cardiomyocytes contractile function is alleviated by more than 50%, e.g., at least about 60%, or at least about 70%. In another embodiment, the cardiomyocytes contractile function is alleviated by at least about 80%, at least about 90% or greater, as compared to a subject having the same disease that was not treated by an inhibitor of bZIP repressor and in particular was not treated by a combination of an inhibitor to ATF3 and an inhibitor to JDP2.

In some embodiments of the invention, the treatment of the invention is considered to be pharmaceutically effective if the dosage alleviates the contractile function of the cardiac sarcomere in at least about 10%, at least about 15%, at least about 20%, at least about 30%, at least about 40%, or at least about 50%. In one embodiment, the contractile function of the cardiac sarcomere is alleviated by more than 50%, e.g., at least about 60%, or at least about 70%. In another embodiment, the contractile function of the cardiac sarcomere is alleviated by at least about 80%, at least about 90% or greater, as compared to a subject having the same disease that was not treated by an inhibitor of bZIP repressor and in particular was not treated by a combination of an inhibitor to ATF3 and an inhibitor to JDP2.

In some embodiments of the invention, the potential small molecules inhibitors maybe screened using a reporter of ATF3 and/or JDP2 activity. This can be done, for example, by using a reporter cell line designed to report for bZIP repression activity using a luciferase reporter. Such a reporter has a basal activity which is dampened by a JDP2 and/or ATF3 activity. Small molecule that is able to suppress bZIP activity is expected to relief the luciferase activity up to the level presented by the reporter cell line in the absence of either JDP2 or ATF3 expression. The small molecule inhibitor can function through several mechanisms including inhibition of the association of the bZIP repressor with their cognate DNA binding elements, prevent homo and hetero dimerization, or prevent association with histone deacetylase proteins (HDAC).

### EXAMPLES

### Materials and Methods

### Mice

All animal studies have been approved by the Technion animal ethics committee and have therefore been performed in accordance with the ethical standards laid down in the 1964 Declaration of Helsinki and its later amendments. This study was carried out in strict accordance with the Guide for the Care and Use of Laboratory Animals of the National Institute of Health. In addition, the protocol was approved by the Committee of the Ethics of Animal Experiments of the Technion. All surgeries were performed under isoflurane anesthesia and all efforts were made to minimize mice suffering using Buprenorphine injection post-surgery (120 μg/Kg). The ATF3 gene is located on chromosome 1, whereas the JDP2 gene is located on chromosome 12. C57BL/6 mice with whole-body ATF3-KO and JDP2-KO were crossed in a ratio of female:male=2:1. This enabled the generation of double knock-out mice (designated hereafter dKO). The dKO mice were born in a Mendelian distribution, and display no overt phenotype. Male and female mice were used in all the experiments performed in this study and analyzed separately.

### TAC Surgery

All experimental protocols were approved by the Institutional Committee for Animal Care and Use at the Technion, Israel Institute of Technology, Faculty of Medicine, Haifa, Israel. All study procedures were complied with the Animal Welfare Act of 1966 (P.L. 89-544), as amended by the Animal Welfare Act of 1970 (P.L.91-579) and 1976 (P.L. 94-279). Transverse aortic constriction (TAC) surgery was performed on male and female Wild type (WT) and dKO mice (10-12 weeks old). All TAC procedures along this study were performed by a single person blinded to the mice genotype.

### Magnetic Resonance Imaging (MRI) Acquisition and Analysis

Cardiac MRI was performed to measure cardiac function and determine the severity of the TAC surgery. Details of the MRI and all other related experimental methods were described previously in Kalfon R, Haas T, Shofti R, Moskovitz J D, Schwartz O, Suss-Toby E, et al. c-Jun dimerization protein 2 (JDP2) deficiency promotes cardiac hypertrophy and dysfunction in response to pressure overload. Int J Cardiol. 2017; 249:357-363. EF was calculated as follows: EF (%)=[(LVEDV−LVESV)/LVEDV]*100.

### Echocardiography

Mice were anesthetized with 1% of isoflurane and kept on a 37° C. heated plate throughout the procedure. An echocardiography was performed using a Vevo2100 micro-ultrasound imaging system (VisualSonics, Fujifilm) which was equipped with 13-38 MHz (MS 400) and 22-55 MHz (MS550D) linear array transducers. Those performing echocardiography and data analysis were blinded to the mice genotype. Cardiac size, shape, and function were analyzed by conventional two-dimensional imaging and M-Mode recordings. Maximal left ventricular end-diastolic (LVDd) and end-systolic (LVDs) dimensions were measured in short-axis M-mode images. Fractional shortening (FS) was calculated as follows: FS (%)=[(LVDd−LVDs)/LVDd]×100. All values were based on the average of Tat least five measurements.

### Heart Harvesting

Following eight weeks of TAC, mice were anesthetized, weighed and sacrificed. Hearts were excised, and ventricles were weighed and then divided into three pieces that were used for protein extraction, RNA purification, and histological analysis.

mRNA Extraction

mRNA was purified from ventricles using an Aurum total RNA fatty or fibrous tissue kit (#732-6830, Bio-Rad) according to the manufacturer's instructions.

Quantitative Real Time PCR (qRT-PCR)

cDNA was synthesized from 800 ng of purified mRNA derived from the ventricles. Purified mRNA was added to a total reaction mix of high-capacity cDNA reverse transcription kit (#4368814, Applied Biosystems) in a final volume of 20 μl. Real-time PCR was performed using Rotor-Gene 6000™ (Corbett) equipment with absolute blue SYBR green ROX mix (Thermo Scientific AB-4162/B). Serial dilutions of a standard sample were included for each gene to generate a standard curve. Values were normalized to ubiquitin-conjugating enzyme E2D) 2A (Ube2d2a) expression levels. The primer sequences are shown in Table 1 below.

| Primer | Sequence |
|---|---|
| ATF3 | F- GAGGATTTTGCTAACCTGACACC |
| | (SEQ ID No. 1) |
| | R- TTGACGGTAACTGACTCCAGC |
| | (SEQ ID No. 2) |
| ACTA1 | F- CCCAAAGCTAACCGGGAGAAG |
| | (SEQ ID No. 3) |
| | R- CCAGAATCCAACACGATGCC |
| | (SEQ ID No. 4) |
| ACTA2 | F- GTCCCAGACATCAGGGAGTAA |
| | (SEQ ID No. 5) |
| | R- TCGGATACTTCAGCGTCAGGA |
| | (SEQ ID No. 6) |
| ACTC1 | F- GTGCCAGGATGTGTGACGA |
| | (SEQ ID No. 7) |
| | R- CTGTCCCATACCCACCATGAC |
| | (SEQ ID No. 8) |
| BNP | F- GAGGTCACTCCTATCCTCTGG |
| | (SEQ ID No. 9) |
| | R- GCCATTTCCTCCGACTTTTCTC |
| | (SEQ ID No. 10) |
| αMHC | F- TGCAAAGGCTCCAGGTCTGA |
| | (SEQ ID No. 11) |
| | R- CTTGAACCTGTCCAACCACAA |
| | (SEQ ID No. 12) |
| col 1α | F- CTGGCGGTTCAGGTCCAAT |
| | (SEQ ID No. 13) |
| | R- TTCCAGGCAATCCACGAGC |
| | (SEQ ID No. 14) |
| F4/80 | F- CCCCAGTGTCCTTACAGAGTG |
| | (SEQ ID No. 15) |
| | R- GTGCCCAGAGTGGATGTCT |
| | (SEQ ID No. 16) |
| IL-1β | F- GCAACTGTTCCTGAACTCAACT |
| | (SEQ ID No. 17) |
| | R- ATCTTTTGGGGTCCGTCAACT |
| | (SEQ ID No. 18) |
| IL-6 | F- TAGTCCTTCCTACCCCAATTTCC |
| | (SEQ ID No. 19) |
| | R- TTGGTCCTTAGCCACTCCTTC |
| | (SEQ ID No. 20) |
| JDP2 | F- GAAGAAGAGCGAAGGAAAAGGC |
| | (SEQ ID No. 21) |
| | R- GCATCAGGATAAGCTGTTGCC |
| | (SEQ ID No. 22) |
| TGFβ3 | F- CCTGGCCCTGCTGAACTTG |
| | (SEQ ID No. 23) |
| | R- GACGTGGGTCATCACCGAT |
| | (SEQ ID No. 24) |
| Ube2d2a | F- ACAAGGAATTGAATGACCTGGC |
| | (SEQ ID No. 25) |
| | R- CACCCTGATAGGGGCTGTC |
| | (SEQ ID No. 26) |

### Cell Size Analysis

Heart tissue was fixed in 4% formaldehyde overnight, embedded in paraffin, serially sectioned at 10 μm intervals, and then mounted on slides. Sections were stained following deparaffinization with Wheat-germ agglutinin FITC-conjugated (Sigma Aldrich Cat #L4895) and diluted to a 1:100 phosphate-buffered saline (PBS). Sections were washed three times with PBS and mounted in Fluorescence Mounting Medium (Dako, S3023). Images were acquired by using panoramic flash series digital scanner (3DHistech Pannoramic 250 Flash III). Quantification of the cell size was performed with Image Pro Plus software. Five fields in each slide were photographed. Unstained areas were then identified and segmented using Image Pro Plus software. In each stained area, the mean cell perimeter and area was calculated, and the number of cells was measured.

### Fibrosis Staining

Heart tissue was fixed in 4% formaldehyde overnight, embedded in paraffin, serially sectioned at 10 μm intervals, and then mounted on slides. Masson's trichrome staining was performed according to the standard protocol. Images were acquired by using Virtual Microscopy (Olympus). The percent of the interstitial fibrosis was determined as the ratio of the fibrosis area to the total area of the heart section using Image Pro Plus software.

### Western Blot Analysis and Quantification

Harvested tissues were homogenized in RIPA buffer (PBS containing 1% NP-40, 5 mg/ml Na-deoxycholate, 0.1% SDS) and supplemented with a protease inhibitor cocktail (P-8340, Sigma Aldrich). Homogenization was performed at 4° C. using the Bullet Blender homogenizer (BBX24; Next advance) according to the manufacturer's instructions as previously described (Koren, 2015 #1364).

### Antibodies

The primary antibodies used: anti-phospho-ERK (Cat #M-9692) was purchased from Sigma Aldrich. Anti-p38 (Cat #9212), anti-phospho-p38 (Cat #9211) and anti-ERK (Cat #9102) were purchased from Cell Signaling.

### Statistics

The data in here is expressed as means f SE. The comparison between several means was analyzed by one-way ANOVA followed by Tukey's post hoc analysis. All statistical analyses were performed using GraphPad Prism 5 software (La Jolla, Calif.). A P value≤0.05 was accepted as statistically significant.

### EXPERIMENTAL RESULTS

### Example 1

To test the hypothesis that elevated expression of ATF3 in JDP2-KO mice is responsible for the deteriorated cardiac phenotype following TAC, ATF3 was deleted in the JDP2-KO background by crossing the JDP2 KO with the ATF3-KO mice to generate the whole body dKO mice.

### Analysis of Cardiac Hypertrophy at Basal

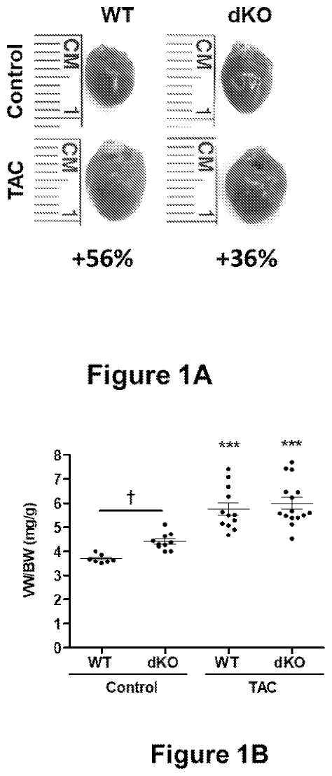

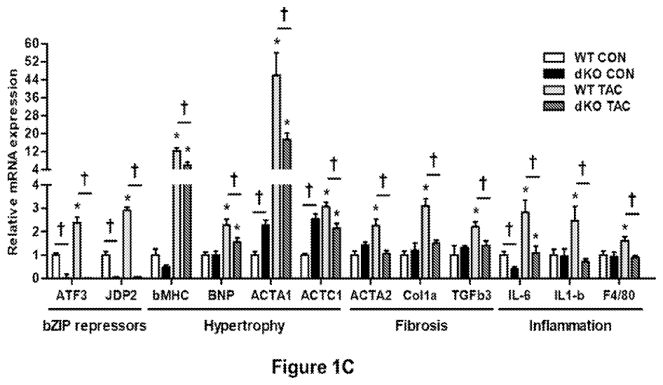

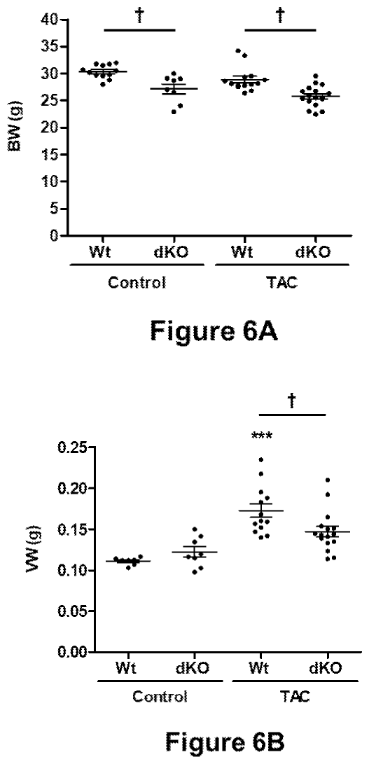

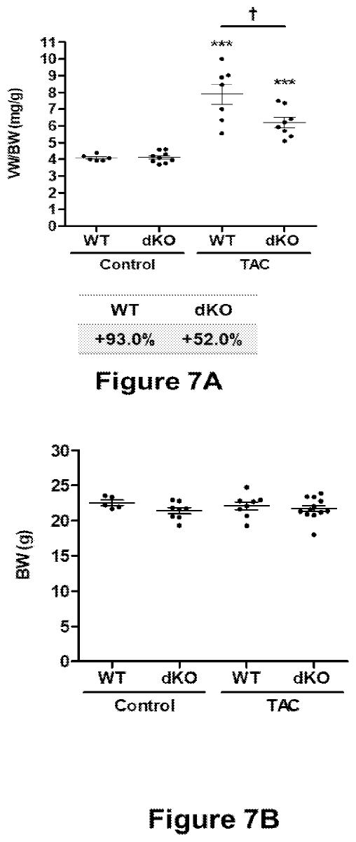

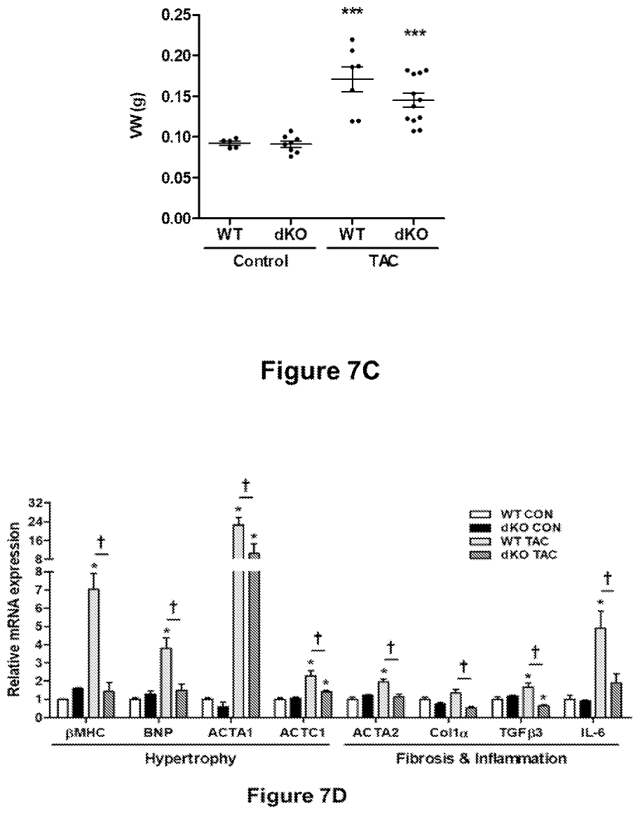

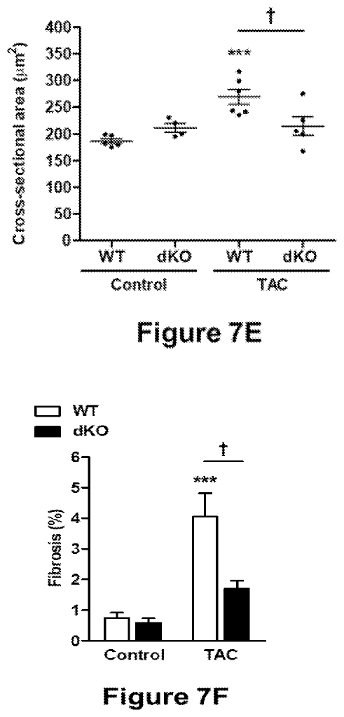

The mice under control (unstressed) condition was examined first. Hearts from 20-weeks-old dKO male mice were bigger in size and had a slightly higher (statistically significant. P≤0.05) ventricular weight/body weight (VW/BW) ratio than the WT male mice (FIG. 1). While the VW of both mice genotypes was not different, the basal BW of dKO mice strain was significantly lower (FIG. 6). Indeed, no significant increase was observed in hypertrophic markers associated with maladaptive remodeling, such as PMHC or BNP (FIG. 1C). Next it was examined whether the higher VW/BW found in dKO male mice is gender-specific by examining the female mice. As shown in FIG. 7, female mice showed no difference in basal VW/BW ratio as well as BW and VW between WT and dKO mice. Thus, only male mice had a slight increase VW/BW ratio, which corresponded mainly to the lower BW. Consistently, the expression levels of two sarcomeric actin isoforms, ACTA1 and ACTC1, were significantly elevated in dKO male mice as compared with their WT counterparts (FIG. 1C), whereas in female hearts the hypertrophic and sarcomeric markers were similar between WT and dKO (FIG. 7).

### Example 2

### Analysis of Cardiac Hypertrophy Following TAC

To test the role of dual deficiency in JDP2 and ATF3 expression in stress-induced cardiac remodeling, 12-week-old mice were exposed to TAC for 8 weeks before analyses. To reveal the potential role of ATF3 and JDP2, their expression levels following TAC was assessed by qRT-PCR (FIG. 1C). As previously shown, both JDP2 and ATF3 expression levels were elevated, whereas, in dKO mice no expression was observed. In males, hearts size and VW/BW ratio were significantly increased in both WT and dKO mice (FIG. 1A, 1B). However, due to the higher basal VW/BW ratio in dKO mice, the calculated percentage increase was higher in WT than dKO mice: 56% versus 36%. (FIG. 1A, B). In female mice, TAC resulted in increased heart size and VW/BW ratio in both genotypes and again with a statistical significant higher impact on the WT than dKO mice: 93% versus 52% (FIG. 7). The increase in heart size following TAC was accompanied by an elevation of hypertrophic markers, such as, OMHC, BNP, ACTA1 and ACTC1 in both genotypes (FIG. 1C and FIG. 7). Consistent with the reduced severe phenotype in dKO, the increase in hypertrophic markers of TAC-operated dKO mice was significantly lower as compared with the WT counterparts in both genders (FIG. 1C and FIG. 7). Interestingly, while in WT male mice the expression of the ACTC1, the abundant cardiac actin isoform, was highly elevated following TAC, no increase in ACTC1 expression was observed in dKO male mice (FIG. 1C). Suggesting that no further increase was necessary in this sarcomeric protein to cope with the pressure overload condition in the dKO mice hearts.

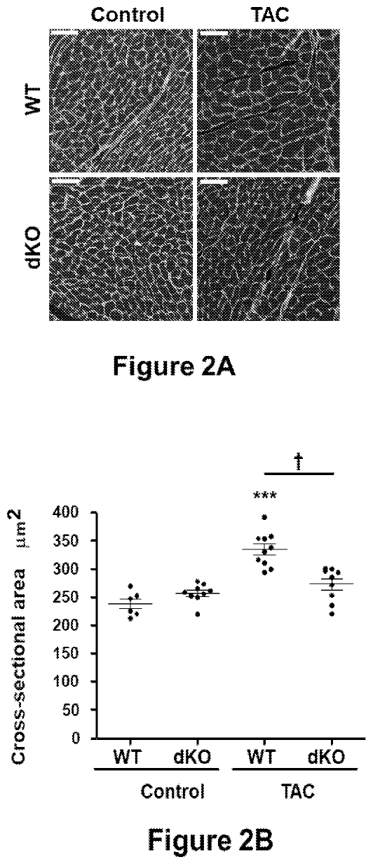

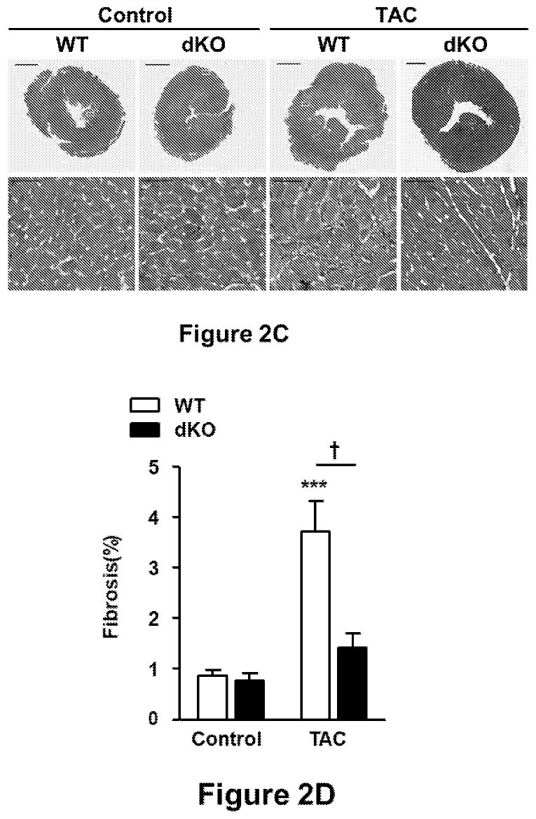

To assess the size of cardiomyocytes following TAC, heart sections were stained by fluorescently labeled wheat germ agglutinin to delineate the cell boundary, and cardiomyocyte cross sectional area (CSA) of control and TAC-operated mice was calculated. In both genders, WT mice showed an increase of cardiomyocyte CSA by about 50% following TAC, but the dKO mice showed no significant increase (FIG. 2A, 2B and FIG. 7).

### Example 3

### Analyses of Fibrosis and Inflammatory Markers

The cardiac fibrosis as part of cardiac remodeling hallmark was next examined. Quantitative analysis of fibrosis showed no difference between the genotypes at baseline (FIG. 2C, 2D and FIG. 7). However, hearts derived from TAC-operated WT mice displayed a 4-fold increase, while dKO mice had only a mild increase (not statistically significant) in cardiac fibrosis (FIG. 2C, 2D). Similar results were observed in female mice (FIG. 7). The increase in fibrosis in TAC-operated WT mice was accompanied by significantly elevated transcripts of fibrosis genes in both genders such as, ACTA2, ColIα and TGFβ3. Consistently, the transcripts of these markers did not increase in TAC-operated dKO mice in both gender (FIG. 1C and FIG. 7).

The inflammatory response of the heart following TAC was next examined by examining IL-6 and IL-1β inflammatory markers, and F4/80, the marker for macrophages. All three markers were lower in TAC-operated dKO male mice than in the WT counterparts (FIG. 1C). The dampened inflammatory response is consistent with the milder hypertrophy and fibrosis observed in dKO mice. Similarly, IL-6 transcription was not elevated in female dKO mice (FIG. 7).

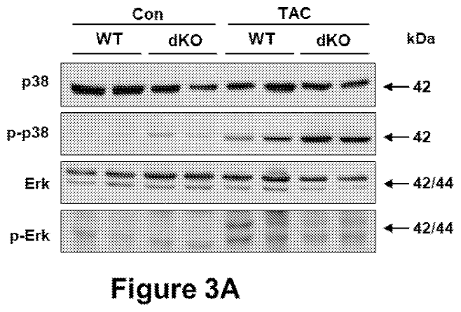

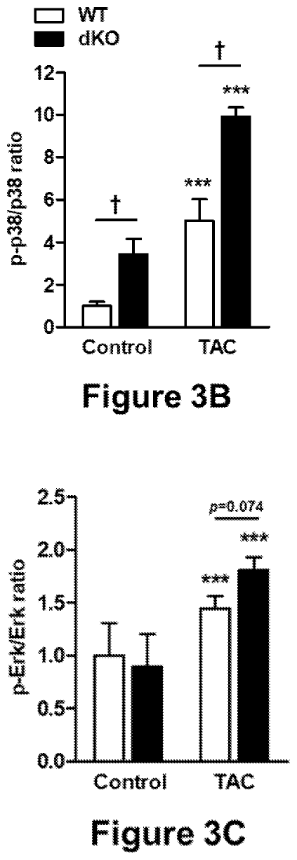

In previous analyses of JDP2 KO mice, the activation of p38 was completely lost following TAC, and this lack of p38 activation correlated with maladaptive cardiac remodeling in these mice. Thus, the activation state of p38 was examined by immunoblot. At baseline, a higher phospho-p38/p38 ratio was observed in the hearts of dKO mice as compared with WT (FIG. 3A, 3B). Following TAC, p38 activation increased in both groups, but was more pronounced in the dKO mice (FIG. 3A, 3B, a 10-fold versus 5-fold increase). Thus, following TAC, the lack of p38 activation, a feature that correlated with maladaptive cardiac remodeling in the hearts of JDP2 KO mice, was fully eliminated in the dKO mice (FIG. 3A, 3B). Interestingly, TAC activated the extracellular regulated kinase (ERK) independent of the WT versus dKO genotype (FIG. 3A, 3C), a result similar to previous data that was independent of single deletion of either ATF3 or JDP2. Thus, these two bZIP genes had no impact on ERK activation by TAC.

### Example 4

Analyses of Cardiac Function: The JDP2/ATF3 dKO Mice Performed Better than the WT Mice Under TAC

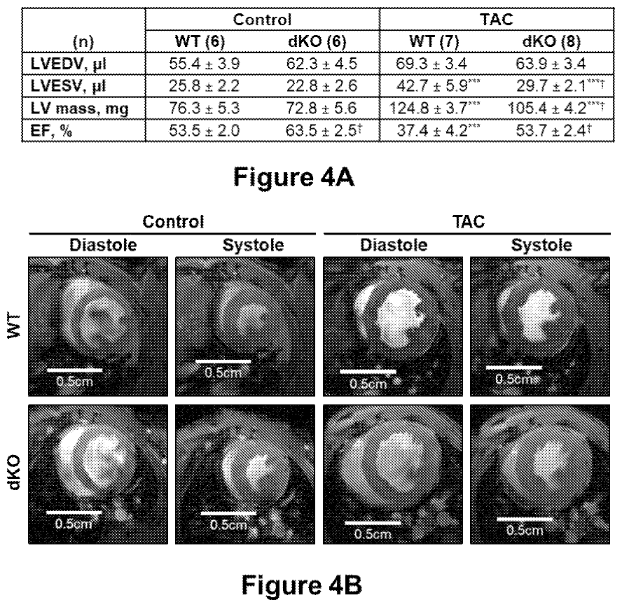

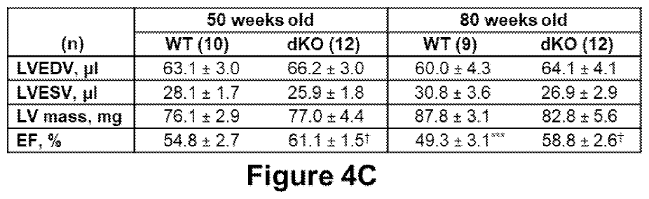

Maladaptive cardiac remodeling characterized by hypertrophy, inflammation and fibrosis is associated with reduced cardiac function. To assess cardiac contractile function, MRI was used to calculate ejection fraction (EF) in control and TAC-operated male mice. The calculated EF in control mice suggests an improved basal contractile function in the dKO mice (higher EF than WT) at 20 weeks of age (FIG. 4A and Table 2, 4B). To assess the long-term effect of JDP2 and ATF3 deficiency on cardiac function, the EF of 50 and 80 weeks old mice were measured. An improvement of 10-20% in the calculated EF in dKO mice was preserved for at least 80 weeks (FIG. 4C and Table 3). Next cardiac volumes, function and mass following TAC were tested. Indeed, TAC induced cardiac morphological changes (as shown by ventricular dilation and increased mass) and led to reduced cardiac function (as shown by reduced EF). However, these changes were quite different between genotypes. Consistent with the greater increase in VW/BW ratio by TAC in the WT male mice, the increase in left ventricular (LV) mass by TAC was significantly higher in the WT mice than that in the dKO mice: 64% versus 45% (FIG. 4A). The hearts derived from TAC-operated WT mice showed a dilated phenotype with LV end diastolic volume (LVEDV) of 69.3 μl after TAC as compared with 55.4 μl at baseline (FIG. 4A and Table 2). In contrast, LVEDV of TAC-operated dKO mice were 63.9 μl, which was very similar to that at baseline: 62.3 μl (FIG. 4A and Table 2). In addition, the LV end systolic volume (LVESV) was significantly increased by TAC in both genotypes; however, the increase was significantly higher in the WT mice than dKO mice (65% versus 30%), indicating that the WT heart was less effective during systole (FIG. 4A). As expected, EF was highly reduced in WT TAC-operated mice as compared to their control counterparts (−30%). Interestingly, TAC-operated dKO mice exhibited only a modest reduction in EF (−15%). In fact, the absolute EF value following TAC of dKO mice was similar to the EF obtained in control (unstressed) WT mice (FIG. 4A and Table 2). Cardiac function in the female mice by echocardiography was examined and the fractional shortening (FS) were calculated.

TABLE 2

| | Control | | TAC | |
|---|---|---|---|---|
| (n) | WT (6) | dKO (6) | WT (7) | dKO (8) |
| LVEDV, μl | 55.4 ± 3.9 | 62.3 ± 4.5 | 69.3 ± 3.4 | 63.9 ± 3.4 |
| LVESV, μl | 25.8 ± 2.2 | 22.8 ± 2.6 | 42.7 ± 5.9*** | 29.7 ± 2.1***^{†} |
| LV mass, mg | 76.3 ± 5.3 | 72.8 ± 5.6 | 124.8 ± 3.7*** | 105.4 ± 4.2***^{†} |
| EF, % | 53.5 ± 2.0 | 63.5 ± 2.5^{†} | 37.4 ± 4.2*** | 53.7 ± 2.4^{†} |

Table 2 demonstrates the following parameters that were measured: left ventricular (LV) mass, left ventricular end-diastolic (LVEDV) and left ventricular end-systolic volume (LVESV), and ejection fraction (EF) was calculated. The results represent the mean±SE of the indicated number (n) of animals per group. ***P≤0.05, control vs. TAC; ^{†}P≤0.05, difference between genotypes.

TABLE 3

| | 50 weeks old | | 80 weeks old | |
|---|---|---|---|---|
| (n) | WT(10) | dKO (12) | WT (9) | dKO (12) |
| LVEDV, μl | 63.1 ± 3.0 | 66.2 ± 3.0 | 60.0 ± 4.3 | 64.1 ± 4.1 |
| LVESV, μl | 28.1 ± 1.7 | 25.9 ± 1.8 | 30.8 ± 3.6 | 26.9 ± 2.9 |
| LV mass, mg | 76.1 ± 2.9 | 77.0 ± 4.4 | 87.8 ± 3.1 | 82.8 ± 5.6 |
| EF, % | 54.8 ± 2.7 | 61.1 ± 1.5^{†} | 49.3 ± 3.1*** | 58.8 ± 2.6^{†} |

Table 3 shows age-related decline in cardiac function as was assessed at 50- and 80-weeks-old mice. Results were compared with control mice (20 weeks old). Left ventricular cardiac volumes, mass and function were examined by a cardiac MRI as described in FIG. 4A. The results represent the mean±SE of the indicated number (n) of animals per group. ***P≤0.05, control vs. TAC; ^{†}P≤0.05, difference between genotypes. ***P≤0.05, different from 20- and 50-weeks-old mice; ^{†}P≤0.05, difference between genotypes.

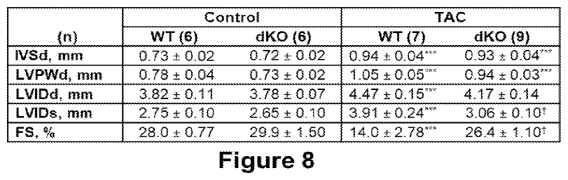

At basal, no significant differences in FS was observed between WT and dKO control mice (FIG. 8 and Table 3 below). Consistent with the male mice findings, it was found that in WT TAC-operated females the FS was highly reduced. It declined from ˜28% to 15%, while in the dKO mice the FS was preserved (from ˜30% to 26%, a reduction with no statistically significance) and indistinguishable from control WT mice (FIG. 8 and Table 4).

TABLE 4

| | Control | | TAC | |
|---|---|---|---|---|
| (n) | WT (6) | dKO (6) | WT (7) | dKO (9) |
| IVSd, mm | 0.73 ± 0.02 | 0.72 ± 0.02 | 0.94 ± 0.04*** | 0.93 ± 0.04*** |
| LVPWd, mm | 0.78 ± 0.04 | 0.73 ± 0.02 | 1.05 ± 0.05*** | 0.94 ± 0.03*** |
| LVIDd, mm | 3.82 ± 0.11 | 3.78 ± 0.07 | 4.47 ± 0.15*** | 4.17 ± 0.14 |
| LVIDs, mm | 2.75 ± 0.10 | 2.65 ± 0.10 | 3.91 ± 0.24*** | 3.06 ± 0.10^{†} |
| FS, % | 28.0 ± 0.77 | 29.9 ± 1.50 | 14.0 ± 2.78*** | 26.4 ± 1.10^{†} |

Table 4 is a table showing that dKO female mice preserve contractile function following TAC. Cardiac hypertrophy was induced by TAC in female mice. Eight weeks following TAC, mice hearts were examined by micro ultrasound. The following parameters were measured: interventricular septal end diastole (IVSd); left ventricular posterior wall end diastole (LVPWd); maximal left ventricular internal end-diastole (LVIDd); end-systole (LVIDs); and fractional shortening (FS). FS was assessed according to: FS (%)=[(LVDd-LVDs)/LVDd] *100. All results represent the means f SE of the indicated number (n) of animals per group. ***P≤0.05, control vs. TAC; ^{†}P≤0.05, difference between genotypes.

Collectively, following TAC, the hearts derived from both WT and dKO mice underwent hypertrophy, yet, the hearts derived from dKO mice showed reduced cardiac hypertrophy and suppressed maladaptive remodeling processes with highly preserved contractile function as compared with WT mice in both genders.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treating a cardiovascular disease in a subject in need comprising the step of administering an inhibitor of bZIP repressor or an activator of p38 or a combination thereof to a subject in need thereby treating the cardiovascular disease.

2. The method of claim 1, wherein the inhibitor to bZIP repressor is:
an inhibitor of ATF3;
an inhibitor of JDP2;
a co-inhibitor to both ATF3 and JDP2; or
a combination of an inhibitor of ATF3 and an inhibitor of JDP2.

3. The method of claim 2, wherein the inhibitor to ATF3 and the inhibitor to JDP2 are administered simultaneously or sequentially.

4. The method of claim 1, wherein the inhibitor is a protein, a peptide, a small molecule or an agent, which prevents or reduces the expression of the bZIP repressor.

5. The method of claim 1, wherein the activator of p38 is a protein, a peptide, a small molecule or an agent, which increases the activity of the p38.

6. The method of claim 4, wherein the agent which decreases the expression of the bZIP repressor is an inhibitor of the mRNA encoding the bZIP repressor.

7. The method of claim 6, wherein the inhibitor of the mRNA encoding the bZIP repressor is an antisense RNA, triple helix molecule, ribozyme, microRNA, or siRNA that recognizes the bZIP repressor mRNA.

8. The method of claim 5, wherein the agent which increases the expression of the p38 is an mRNA encoding the p38 or an activator thereof.

9. The method of claim 6, wherein the activator of the mRNA encoding the p38 or the activator thereof is an antisense RNA, triple helix molecule, ribozyme, microRNA, or siRNA that recognizes the bZIP repressor mRNA.

10. The method of claim 1, wherein the cardiovascular disease is heart failure.

11. The method of claim 10, wherein the heart failure is a chronic heart failure (CHF).

12. The method of claim 1, wherein the cardiovascular disease is accompanied by maladaptive cardiac remodeling process.

13. The method of claim 1, wherein the cardiovascular disease is accompanied by reduced contractile function.

14. The method of claim 1, wherein the cardiovascular disease is accompanied by maladaptive cardiac remodeling process.

15. The method of claim 1, wherein the cardiovascular disease is accompanied by reduced contractile function.

16. The method of claim 1, wherein the treating is effected by improvement of the contraction of the cardiomyocyte.
